(19) United States
(12) Patent Application Publication
(10) Pub. No.: US 2019/0105277 A1
(43) Pub. Date: Apr. 11, 2019

(54) PHARMACEUTICAL COMPOSITION COMPRISING AN ATYPICAL ANTIPSYCHOTIC AGENT AND METHOD FOR THE PREPARATION THEREOF

(71) Applicant: PHARMATHEN S.A., Pallini Attikis (GR)

(72) Inventors: Evangelos KARAVAS, Pallini Attikis (GR); Efthimios KOUTRIS, Pallini Attikis (GR); Vasiliki SAMARA, Pallini Attikis (GR); Ioanna KOUTRI, Pallini Attikis (GR); Lida KALANTZI, Pallini Attikis (GR); Anastasia KALASKANI, Pallini Attikis (GR); Christina KIZIRIDI, Pallini Attikis (GR); Morfis ABATZIS, Pallini Attikis (GR)

(73) Assignee: PHARMATHEN S.A., Pallini Attikis (GR)

(21) Appl. No.: 16/090,915

(22) PCT Filed: Apr. 5, 2016
(86) PCT No.: PCT/EP2016/000555

§ 371 Date: Oct. 3, 2018

(51) Int. Cl. A61K 9/48; A61K 9/20; A61K 9/28; A61K 31/519

(52) U.S. Cl. CPC A61K 9/4808; A61K 9/2077; A61P 25/18; A61K 9/2866; A61K 31/519; A61K 9/2846

(57) ABSTRACT

The present invention relates to controlled release pharmaceutical formulations comprising an atypical antipsychotic agent such as Paliperidone in the form of capsule filled with mini-tablets that provide zero order drug release. It also relates to a process for the preparation thereof.

### TECHNICAL FIELD OF THE INVENTION

The present invention relates to oral dosage forms for the controlled delivery of an atypical antipsychotic agent such as Paliperidone and a method for the preparation thereof.

### BACKGROUND OF THE INVENTION

Antipsychotics are the mainstay of treatment of schizophrenia. Conventional antipsychotics, typified by haloperidol, have a proven track record over the last half-century in the treatment of schizophrenia. While these drugs are highly effective against the positive, psychotic symptoms of schizophrenia, they show little benefit in alleviating negative symptoms or the cognitive impairment associated with the disease.

Second generation antipsychotics such as Paliperidone, also called atypical antipsychotics, differ considerably in their chemical, pharmacological, and clinical profiles and are generally characterized by effectiveness against both positive and negative symptoms associated with schizophrenia and by enhanced safety profile with respect to extrapyramidal symptoms.

Paliperidone also known as 9-hydroxyrisperidone is the major metabolite of risperidone. It shares the characteristic serotonin (5HT_{2A}) and dopamine (D₂) antagonism and receptor binding profile of its parent risperidone. It binds also to a₁-adrenergic receptors, and, with lower affinity, to H₁-histaminergic and a₂-adrenergic receptors, which may explain some of the other effects of Paliperidone.

The chemical name of Paliperidone is 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one. The molecular formula is C₂₃H₂₇FN₄O₃ corresponding to a molecular weight of 426.48. It is a white to yellow color, non-hygroscopic powder. Paliperidone is sparingly soluble in dichloromethane, slightly soluble in 0.1N HCl and insoluble in water.

WO A-2007/044234 discloses an osmotic dosage form comprising a semi-permeable membrane, a first and a second orifice in the semi-permeable membrane, a controlled release drug layer, a push layer, a fast release drug layer and a barrier layer.

EP B 1539115 discloses a dosage form comprising two or more layers, said first layer comprises Paliperidone, said second layer comprises a polymer, an outer wall surrounding said two or more layers and an orifice in said outer wall.

Although each of the patents above represents an attempt to provide dosage forms for the sustained delivery of Paliperidone, there still exists a need for alternative means of controlling delivery that would enhance the initial tolerability and permit initiation of treatment at an effective dose without the need for initial dose titration.

### SUMMARY OF THE INVENTION

It is, therefore, an object of the present invention to provide a stable oral solid dosage formulation comprising an atypical antipsychotic agent and in particular Paliperidone, as an active ingredient, providing a uniform and constant rate of release over an extended period of time.

It is another object of the present invention to provide a controlled release pharmaceutical composition for oral administration comprising Paliperidone as an active ingredient, which is bioavailable, with sufficient self-life and good pharmacotechnical properties.

The present invention's target is to provide a delayed release medicament in the form of a hard gelatin capsule filled with mini tablets. The use of mini tablets is beneficial because it permits the release of the active ingredient at different sites along the gastrointestinal tract.

A major object of the present invention is to provide a matrix controlling core comprising specific quantity of hydrophilic/swelling polymer and insoluble/hydrophobic polymer.

An essential object of the present invention is to choose a coating technology which in combination with matrix controlling core will allow drug release with zero order kinetics.

A further approach of the present invention is to provide a controlled release dosage form containing Paliperidone which is manufactured through a fast, simple and cost-effective process.

Other objects and advantages of the present invention will become apparent to those skilled in the art in view of the following detailed description.

### DETAILED DESCRIPTION OF THE INVENTION

For the purposes of the present invention, a pharmaceutical composition comprising an active ingredient (e.g. Paliperidone) is considered to be “stable” if said ingredient degrades less or more slowly than it does on its own and/or in known pharmaceutical compositions.

As already mentioned the main object of the present invention is to provide a controlled release composition of Paliperidone that is simple to manufacture, bioavailable, cost effective, stable and possesses good pharmacotechnical properties.

Paliperidone exhibits polymorphism. Two polymorphs are observed, polymorph I and II, in addition to a hydrate and a solvate. Polymorph I, disclosed in WO-A-2008/021342 entitled “Crystal forms of 9-hydroxy-risperidone”, is used in the present invention as it is the thermodynamically stable crystal form.

The development of the solid dosage form of Paliperidone SR is based on combination of a core that has a matrix controlling the release of Paliperidone and coating technology that also provides additional control of the release of Paliperidone.

The matrix core tablets contain both hydrophilic/swelling polymer and hydrophobic/pH independent polymer commonly used as matrix forming materials for extended release formulations.

The mechanism of drug release from hydrophilic matrix tablets is mainly based on the diffusion of the drug through the hydrated portion of the matrix as well as on the erosion of the outer hydrated polymer on the surface of the matrix. The overall drug release is affected by the rate of water uptake and the diffusion rate of the drug through the swollen gel. Incorporation of a water-insoluble hydrophobic polymer may circumvent the burst release as the former decrease the water penetration in the matrix providing appropriate drug diffusion decrease.

It has been surprisingly found that when hydrophilic and hydrophobic polymers are incorporated together in certain quantities in the tablet matrix of sustained release tablet and the appropriate coating technology is applied then zero order release can be achieved. More particularly, the target is achieved when 30-60% by weight of hydrophilic polymer and 5-10% by weight of hydrophobic polymer of the total weight of uncoated tablet is comprised in matrix of Paliperidone sustained release tablet. The amount of the rest of excipients is 30-65% by weight of the total weight of uncoated tablet.

Hydrophilic polymers that may be used in the present invention are selected from xanthan gum, guar gum, carrageenan, dextran, polyacrylic acid, polyethylene glycol, polyvinylalcohol, polyvinylpyrrolidone, hydroxypropylmethylcellulose (HPMC), hydroxypropylcellulose (HPC), sodium carboxymethylcellulose, hydroxyethylcellulose. HPMC K100M is preferably used in the present invention in a range from 30-60% by weight.

Hydrophobic polymers that may be used in the present invention are selected from polyvinylacetate, polyethylene vinyl acetate, poly(ε-caprolactone), poly(ethylacrylate-methylmethacrylate) such as Eudragit NE30D, ammonio methacrylate copolymers such as Eudragit RL, Eudragit RS. Eudragit RS is preferably used in the present invention in a range from 5-10% by weight.

In addition, for the coating technology it is preferred to use of an enteric coating layer which is preferably an anionic pH-dependent polymer in order to prevent drug release in the stomach and deliver the appropriate amount of drug to specific sites of the GI tract.

pH-dependent polymers used as enteric coating layer in the present invention are selected from methacrylic acid copolymers such as Eudragit L12.5, Eudragit L100 that dissolve in pH>6, Eudragit S12.5, Eudragit S100 that dissolve in pH>7, aqueous dispersions of anionic copolymer based on methacrylic acid and ethyl acrylate such as Kollicoat MAE 100P that dissolves in pH>5.5. Eudragit S100 is preferably used in the present invention in a range from 10-15%.

The pharmaceutical compositions of the present invention may also contain one or more additional formulation excipients such as diluents, lubricants, glidants and antioxidant agents, provided that they are compatible with the active ingredient of the composition, so that it does not interfere with it in the composition and in order to increase the stability of the drug and the self-life of the pharmaceutical product.

Diluents may be, for example, microcrystalline cellulose, dextrates, dextrose, fructose, mannitol, sorbitol, starch, pregelatinized starch, sucrose, xylitol, maltose, maltodextrin, maltitol, microcelac, calcium carbonate.

Antioxidants may be, for example butylated hydroxytoluene (BHT), fumaric acid, sodium ascorbate.

At least a lubricant is incorporated into the formulation to prevent the powder from adhering to tablet punches during the compression procedure. Lubricants may be, for example, magnesium stearate, calcium stearate, glyceryl behenate, hydrogenated castor oil, stearic acid, stearic acid.

Glidants are used to promote powder flow by reducing interparticle friction and cohesion. These are used in combination with lubricants as they have no ability to reduce die wall friction. Glidants, may be, for example, colloidal silicon dioxide, calcium silicate, calcium phosphate tribasic, starch, talc.

By controlling the amount of each individual excipient and thus the extent of its contribution in sustained release properties, the aim is to produce a composition in which the in vitro dissolution profile of Paliperidone will be segmented to achieve minimum release (up to 10%) in the upper GI tract where solubility and permeability are high, about half of the amount released after 12 hours and maximum release over 24 hours since absorption is much decreased in the colon resulting thus in zero order drug release.

Multiple unit dosage forms (MUDFs) like mini-tablets are characterized by the fact that the dose is administered as a number of subunits, each single unit containing the drug. The overall dose is then, the sum of the quantity of the drug in each subunit, and the functionality of the entire dose is directly related to the functionality of the individual subunits.

Mini-tablets have many advantages over single unit dosage forms such as tablets. They can be manufactured relatively easily, they offer flexibility during the formulation development; they have excellent size uniformity, regular shape and a smooth surface, thereby act as an excellent coating substrate; they have less risk of dose dumping; they have less inter and intra-subject variability, they offer high degree of dispersion in the GI tract, thus minimizing the risks of high local drug concentrations; they offer high drug loading, a wide range of release rate patterns and also fine tuning of these release rates.

The development strategy is based on the design of a hard gelatin capsule filled with 2 or 3 different mini-tablets in such way that each tablet delivers the proper amount of drug in the GI tract in certain time intervals and on specific site in order to achieve zero order drug release profile.

Each of the mini tablets follow a different release mechanism, contain different amount of API and have different target for delivery of the drug in the GI tract.

In particular, the first mini tablet contains up to 10% by weight of the total amount of API and is formulated to give a rapid release of the API in the stomach and the upper GI tract where the absorption is complete.

The second mini tablet is a sustained release tablet containing up to 60% by weight of the API and also coated with enteric coating layer to avoid any release in the stomach. The tablet core is a matrix with hydrophilic and hydrophobic polymers and is designed to provide controlled release of the API with zero order.

The third tablet containing the rest amount of API is designed to deliver the API to the colon without any release in stomach or small intestine. Due to the fact that permeability of Paliperidone is very low in the colon, all the API that is delivered has to be dissolved from which only half of it can be absorbed. This can be achieved by the use of pH dependent coating layer which dissolves in pH>7 so that the tablet remains intact throughout the stomach and the small intestine and upon reaching the colon to release the API in a controlled rate and not immediately to avoid any burst release of the API earlier in the small intestine.

All tablet cores have the same size (5 mm or 6 mm round tablets) and approximately the same weight.

For the preparation of the 1^{st} tablet containing 10% by weight API, two different hydrophilic polymers were tested in low amounts so that complete release of the API is achieved up to 3 hours in a controlled rate avoiding a burst release. Microcelac was used as diluent and hydroxypropyl cellulose or hydroxypropyl methyl cellulose as hydrophilic polymers. (Table 1)

TABLE 1
Compositons 1A to 1D

Composition

| | | | |
|---|---|---|---|
| 1A | 1B | 1C | 1D |

%

| Internal phase | | | | |
|---|---|---|---|---|
| Paliperidone | 0.19 | 0.19 | 0.19 | 0.19 |
| Microcelac | 92.76 | 87.76 | 82.76 | 82.76 |
| HPMC K4M | 5.00 | 10.00 | 15.00 | — |
| HPC | — | — | — | 15.00 |
| BHT | 0.05 | 0.05 | 0.05 | 0.05 |
| Aerosil | 1.00 | 1.00 | 1.00 | 1.00 |
| External phase | | | | |
| Magnesium Stearate | 1.00 | 1.00 | 1.00 | 1.00 |

| | |
|---|---|
| Total weight | 100.00 |

Compositions 1A to 1D were prepared according to the following manufacturing process:
Mixing of Paliperidone with microcelac geometrically;
Addition of HPC or HPMC, BHT and aerosil and mixing;
Kneading with ethanol;
Drying of the granules at 40° C.;
Lubrication of the granules with magnesium stearate;
Compression to tablets.

TABLE 2
Dissolution profiles of Compositions 1A to 1D

% release in pH 1.2, 50 rpm

| Time (h) | 1A | 1B | 1C | 1D |
|---|---|---|---|---|
| 0.5 | 10.01 | 2.50 | 2.81 | 2.93 |
| 1.0 | 10.05 | 6.42 | 5.15 | 6.45 |
| 1.5 | 10.05 | 7.30 | 5.96 | 7.63 |
| 2.0 | 10.10 | 8.61 | 7.04 | 8.78 |
| 3.0 | 10.01 | 9.98 | 8.91 | 10.15 |

Based on the results Composition 1D containing hydroxypropyl cellulose in amount of 15% was selected as the most suitable formulation for the 1^{st} tablet providing complete release of the API in a fast but controlled rate.

The tablet matrix core of the 2^{nd} tablet was prepared with HPMC K100M as hydrophilic/gelling agent and Eudragit RS as insoluble/hydrophobic polymer and the amount of API was 50% of the labelled amount. In order to select the proper ratio different amounts of these polymers were tested. (Table 3)

TABLE 3
Compositions 2A to 2J

| | Composition | | | | | | | | |
|---|---|---|---|---|---|---|---|---|---|
| | 2A | 2B | 2C | 2D | 2E | 2F | 2G | 2H | 2J |
| | % | | | | | | | | |
| Internal phase | | | | | | | | | |
| Paliperidone | 0.94 | 0.94 | 0.94 | 0.94 | 0.94 | 0.94 | 0.94 | 0.94 | 0.94 |
| Microcelac | 61.25 | 46.25 | 31.25 | 55.25 | 41.25 | 25.25 | 45.25 | 30.25 | 15.25 |
| HPMC K100M | 30.00 | 45.00 | 60.00 | 30.00 | 45.00 | 60.00 | 30.00 | 45.00 | 60.00 |
| Eudragit RS PO | 5.00 | 5.00 | 5.00 | 10.00 | 10.00 | 10.00 | 20.00 | 20.00 | 20.00 |
| BHT | 0.81 | 0.81 | 0.81 | 0.81 | 0.81 | 0.81 | 0.81 | 0.81 | 0.81 |
| Aerosil | 1.00 | 1.00 | 1.00 | 1.00 | 1.00 | 1.00 | 1.00 | 1.00 | 1.00 |
| External phase | | | | | | | | | |
| Magnesium Stearate | 1.00 | 1.00 | 1.00 | 1.00 | 1.00 | 1.00 | 1.00 | 1.00 | 1.00 |

| | |
|---|---|
| Total weight | 100.00 |

Compositions 2A to 2J were prepared according to the following manufacturing process:
Mixing of Paliperidone with HPMC K100M geometrically;
Addition of microcelac, Eudragit RS and BHT and mixing;
Kneading with solution of ethanol/water;
Drying of the granules at 40° C.;
Lubrication of the granules with magnesium stearate;
Compression to tablets.

The formulations were tested for their dissolution profiles in USP II apparatus, pH 6.8, 150 rpm and data of 12h and 24h % drug release were used as responses to determine the proper ratio of hydrophilic and hydrophobic polymer. The target was to achieve more than 50% drug release at 12h and more than 90% at 24h. According to the results high amount of Eudragit RS seems to retard the drug release especially when low amount of HPMC is used. In order to be closer to the target values of drug release after 12h and 24h, the amount of Eudragit RS has to be limited between 5-10% in combination with amount of HPMC between 30-45%.

Based on the above, Composition 2A was selected as the most proper formulation for the sustained release tablet containing 30% HPMC and 5% Eudragit RS in the matrix core. Tablets of Composition 2A were further coated with enteric polymer (Kollicoat MAE 100P) in order to prevent any drug release at the stomach. The dissolution profile of the coated sustained release tablet is provided in Table 4.

TABLE 4
Dissolution profile of Composition
2A coated with Kollicoat MAE 100P

| | % release in USP II, pH 6.8, 150 rpm |
|---|---|
| Time (h) | 2A coated with Kollicoat MAE 100P |
| 1.0 | 1.28 |
| 2.0 | 2.92 |
| 3.0 | 7.93 |
| 4.0 | 11.20 |
| 6.0 | 16.23 |
| 8.0 | 21.34 |
| 12.0 | 29.61 |
| 14.0 | 33.47 |
| 18.0 | 39.01 |
| 24.0 | 45.34 |

For the preparation of the colon targeted tablet (3^{rd} tablet) the core was prepared based on the 1^{st} tablet core using HPMC K4M at concentration of 15% in order to achieve complete drug release in fast and controlled manner and containing 40% of API.

The tablets were further coated with the enteric polymer Eudragit S100 which dissolves in pH>7. To determine the proper amount of enteric polymer that is sufficient to prevent any drug release throughout the stomach and small intestine and deliver the drug in the colon different amounts of enteric polymer were applied ranging from 5 to 15% and the tablets were tested for their dissolution properties in USP II apparatus, in buffer pH 1.2 for 2h, pH 6.8 for 2 h, pH 7.4 for 2h and pH 6.8 till 24h with 50 rpm.

TABLE 5
Compositions 3A to 3E of colon targeted tablet

Composition

| | | | | |
|---|---|---|---|---|
| 3A | 3B | 3C | 3D | 3E |

%

| Internal phase | | | | | |
|---|---|---|---|---|---|
| Paliperidone | 0.75 | 0.75 | 0.75 | 0.75 | 0.75 |
| Microcelac | 82.20 | 82.20 | 82.20 | 82.20 | 82.20 |
| HPMC K4M | 15.00 | 15.00 | 15.00 | 15.00 | 15.00 |
| BHT | 0.05 | 0.05 | 0.05 | 0.05 | 0.05 |
| Aerosil | 1.00 | 1.00 | 1.00 | 1.00 | 1.00 |
| External phase | | | | | |
| Magnesium Stearate | 1.00 | 1.00 | 1.00 | 1.00 | 1.00 |
| Total tablet weight | 100.00 | | | | |
| Eudragit S100 | 5.00 | 7.00 | 10.00 | 12.00 | 15.00 |

Compositions 3A to 3E were prepared according to the following manufacturing process:
Mixing of Paliperidone with microcelac geometrically;
Addition of HPMC, BHT and aerosil and mixing;
Kneading with ethanol;
Drying of the granules at 40° C.;
Lubrication of the granules with magnesium stearate;
Compression to tablets;
Enteric coating with aqueous dispersion of Eudragit S100 (Eudragit FS 30D).

TABLE 6
Dissolution profile of Compositions 3A-3E

| | % release in pH 1.2 2 h, pH 6.8 2 h, | | | | |
|---|---|---|---|---|---|
| | pH 7.4 2 h, pH 6.8 till 24 h 50 rpm | | | | |
| Time (h) | 3A | 3B | 3C | 3D | 3E |
| 1.0 | 0.32 | 0.32 | 0.32 | 0.28 | 0.15 |
| 2.0 | 0.33 | 0.40 | 0.43 | 0.64 | 0.64 |
| 3.0 | 0.77 | 0.65 | 0.65 | 0.42 | 0.52 |
| 4.0 | 2.28 | 0.66 | 0.66 | 0.64 | 0.81 |
| 6.0 | 5.96 | 9.44 | 3.67 | 1.85 | 1.18 |
| 8.0 | 7.21 | 23.48 | 16.54 | 11.26 | 9.95 |
| 12.0 | 19.80 | 36.87 | 36.12 | 30.39 | 31.28 |
| 14.0 | 29.79 | 37.30 | 39.54 | 36.15 | 35.64 |
| 18.0 | 32.48 | 38.33 | 39.89 | 38.45 | 36.87 |
| 24.0 | 33.90 | 39.69 | 41.57 | 39.46 | 39.46 |

Based on the dissolution results it can be concluded that amounts of enteric polymer lower than 10% are not able to prevent the release at pH 7.4 resulting in drug release in the small intestine and not in the colon. Thus, Composition 3D was selected as the optimum formulation with 12% enteric polymer in the coating layer which is effective in protecting the API even in pH 7.4 without compromising the release after 24h.

The three tablets selected during the formulation development were combined in the final dosage form, i.e. 3 mini-tablets in hard gelatin capsule. The final formulation was tested in sequential dissolution media due to the pH-dependent release mechanism used in the third tablet. (Table 7)

TABLE 7
Dissolution profile of 3 mini tablets in hard gelatin capsule (test 1)

% release of test 1

| | 2 h pH 1.2- 2 h 6.8- | 2 h pH 1.2- 2 h 6.8- |
|---|---|---|
| | 2 h pH 7.4- 18 h pH 6.8, | 2 h pH 7.4- 18 h pH 6.8, |
| Time (h) | 150 rpm | 50 rpm |
| 1.0 | 8.24 | 6.66 |
| 2.0 | 10.07 | 8.79 |
| 3.0 | 17.37 | 14.46 |
| 4.0 | 19.88 | 22.23 |
| 6.0 | 26.27 | 25.55 |
| 8.0 | 44.25 | 35.61 |
| 12.0 | 73.83 | 57.98 |
| 14.0 | 82.73 | 66.58 |
| 18.0 | 89.46 | 77.19 |
| 24.0 | 96.16 | 89.46 |

The formulation was also tested in vivo and according to its plasma concentration profile the test 1 composition provides a faster and much higher absorption profile than the required one and it was attributed to the fast release tablet (1^{st} tablet) which contains 10% of the total amount of API and is completely absorbed. The entire amount that is dissolved in the stomach and in the upper small intestine is completely absorbed. Also, the controlled release tablet (2^{nd} tablet) contributed to the high absorption rate and extent and thus a slower drug release had to be achieved.

Based on the above conclusions, the final dosage form was designed as a capsule containing two mini tablets having excluded the fast release tablet, in which the first tablet containing 60% of the API provided the sustained release of the API and the second tablet with 40% API was coated with pH-dependent polymer in order to release the API in the colon.

For the preparation of the sustained release tablet it was considered necessary to increase the amount of the hydrophilic and/or the hydrophobic polymer in order to retard the release rate of the API from the matrix. In addition, microcelac was replaced with microcrystalline cellulose which is insoluble to further retard the release rate. The composition and the dissolution profile of the sustained release tablet are presented in Tables 8 & 9.

TABLE 8
Compositions 4A-4D of sustained release tablet

Composition

| | | | |
|---|---|---|---|
| 4A | 4B | 4C | 4D |

%

| | | | | |
|---|---|---|---|---|
| Internal phase | | | | |
| Paliperidone | 0.72 | 0.72 | 0.72 | 0.72 |
| Microcrystalline | 56.47 | 46.47 | 31.47 | 41.47 |
| cellulose | | | | |
| HPMC K100M | 30.00 | 45.00 | 60.00 | 45.00 |
| Eudragit RS PO | 10.00 | 5.00 | 5.00 | 10.00 |
| BHT | 0.81 | 0.81 | 0.81 | 0.81 |
| Aerosil | 1.00 | 1.00 | 1.00 | 1.00 |
| External phase | | | | |
| Magnesium Stearate | 1.00 | 1.00 | 1.00 | 1.00 |

| | |
|---|---|
| Total tablet weight | 100.00 |

| | | | | |
|---|---|---|---|---|
| Kollicoat MAE 100P | 2.00 | 2.00 | 2.00 | 2.00 |

Compositions 4A to 4D were prepared according to the following manufacturing process:
Mixing of Paliperidone with HPMC K100M geometrically;
Addition of MCC, Eudragit RS and BHT and mixing;
Kneading with ethanol;
Drying of the granules at 40° C.;
Lubrication of the granules with magnesium stearate;
Compression to tablets;
Coating the tablets with Kollicoat MAE100P.

TABLE 9
Dissolution profile of Compositions 4A-4D

% release in pH 6.8 150 rpm

| Time (h) | 4A | 4B | 4C | 4D |
|---|---|---|---|---|
| 1.0 | 5.68 | 5.60 | 4.88 | 4.73 |
| 2.0 | 8.78 | 8.63 | 7.66 | 7.94 |
| 3.0 | 12.23 | 11.59 | 10.27 | 10.61 |
| 4.0 | 15.47 | 15.22 | 13.16 | 13.35 |
| 6.0 | 21.26 | 20.53 | 17.60 | 18.57 |
| 8.0 | 26.53 | 25.55 | 22.55 | 22.71 |
| 12.0 | 34.85 | 35.36 | 30.42 | 31.30 |
| 14.0 | 39.55 | 39.17 | 35.24 | 35.71 |
| 18.0 | 45.28 | 46.71 | 43.93 | 43.12 |
| 24.0 | 51.64 | 51.74 | 48.46 | 46.84 |

According to the results, Composition 4C containing 60% HPMC K100M and 5% Eudragit RS provided the desirable dissolution profile and was further combined with Composition 3D.

Consequently, the preferred composition of the present invention is a capsule filled with two mini-tablets, a sustained release tablet (Composition 4C) & a colon targeted tablet (Composition 3D).

The dissolution profile of the preferred composition of the present invention is shown in Table 10 below.

TABLE 10
Dissolution profile of the preferred composition

% release of preferred composition

| | 2 h pH 1.2- 2 h 6.8- | 2 h pH 1.2- 2 h 6.8- |
|---|---|---|
| | 2 h pH 7.4- 18 h pH 6.8, | 2 h pH 7.4- 18 h pH 6.8, |
| Time (h) | 150 rpm | 50 rpm |
| 1.0 | 0.40 | 0.85 |
| 2.0 | 0.90 | 0.99 |
| 3.0 | 6.00 | 5.89 |
| 4.0 | 9.10 | 7.86 |
| 6.0 | 13.60 | 10.06 |
| 8.0 | 22.40 | 11.88 |
| 12.0 | 51.20 | 25.65 |
| 14.0 | 69.50 | 35.48 |
| 18.0 | 79.20 | 51.36 |
| 24.0 | 90.10 | 69.49 |

The preferred composition of the present invention was further tested in vivo and the in vivo results provided a satisfactory plasma concentration profile

Overall, by adjusting the incorporated amount of Paliperidone in the mini tablets and selecting the appropriate grade and amount of hydrophilic and hydrophobic polymers as well as pH-dependent polymers, zero order drug release can be achieved.

In order to study the chemical stability of the preferred composition of the present invention, capsules were stored at 25° C./60% RH and 30° C./65% RH for 6 months and were analyzed in terms of impurities (Table 11).

TABLE 11
Stability study of preferred composition

| SPECIFICATION | Zero time | 3 months | 6 months |
|---|---|---|---|
| | STORAGE AT 25° C./60% RH | | |
| Total impurities | 0.60% | 0.62% | 0.61% |
| NMT 2.0% | | | |
| | STORAGE AT 30° C./65% RH | | |
| Total impurities | 0.60% | 0.65% | 0.69% |
| NMT 2.0% | | | |

According to the stability data it can be concluded that the preferred composition of the present invention is chemically stable after storage at 25° C./60% RH and 30° C./65% RH.

While the present invention has been described with respect to the particular embodiments, it will be apparent to those skilled in the art that various changes and modifications may be made in the invention without departing from the spirit and scope thereof, as defined in the appended claims.

1. A controlled release formulation of Paliperidone comprising at least two different distinct units capable of releasing Paliperidone in certain time intervals and at different sites along the gastrointestinal tract in order to achieve zero order drug release profile, wherein the first unit is coated with pH-dependent polymer that dissolves at pH>5.5 and the second unit is coated with pH-dependent polymer that dissolves at pH>7.

2. A controlled release formulation according to claim 1, wherein the distinct units are in the form of mini-tablets.

3. A controlled release formulation according to claim 2, wherein at least one mini-tablet comprises a combination of at least one hydrophilic polymer and at least one hydrophobic polymer in a mini-tablet matrix core.

4. A controlled release formulation according to claim 3, wherein the at least one hydrophilic polymer is selected from the group consisting of xanthan gum, guar gum, carrageenan, dextran, polyacrylic acid, polyethylene glycol, polyvinylalcohol, polyvinylpyrrolidone, hydroxypropylmethylcellulose, hydroxypropylcellulose, sodium carboxymethylcellulose, and hydroxyethylcellulose.

5. A controlled release formulation according to claim 4, wherein the at least one hydrophilic polymer is hydroxypropylmethylcellulose which is present in an amount of 30-60% by weight of the total weight of the mini-tablet.

6. A controlled release formulation according to claim 3, wherein the at least one hydrophobic polymer is selected from the group consisting of polyvinylacetate, polyethylene vinyl acetate, poly(ε-caprolactone), poly(ethylacrylate-methylmethacrylate), and ammonio methacrylate copolymers.

7. A controlled release formulation according to claim 6, wherein the hydrophobic polymer is ammonio methacrylate copolymer which is present in an amount of 5-10% by weight of the total weight of the mini-tablet.

8. A controlled release formulation according to claim 1, wherein the pH-dependent polymer which dissolves at pH>5.5 is an anionic copolymer of methacrylic acid and ethyl acrylate.

9. A controlled release formulation according to claim 1, wherein the pH-dependent polymer which dissolves at pH>7 is a methacrylic acid copolymer.

10. A controlled release formulation according to claim 9, wherein the methacrylic acid copolymer is present in an amount of 10-15% by weight of the total weight of the second unit.

11. A controlled release formulation according to claim 1, wherein the formulation is in the form of mini-tablets filled into a capsule.
